(19) United States
(12) Patent Application Publication
(10) Pub. No.: US 2025/0144077 A1
(43) Pub. Date: May 8, 2025

(54) DOSAGE REGIMEN FOR THROMBOPOIETIN RECEPTOR AGONIST

(71) Applicant: JIANGSU HENGRUI PHARMACEUTICALS CO., LTD., Lianyungang (CN)

(72) Inventors: Hongda LIN, Lianyungang (CN); Kai SHEN, Lianyungang (CN); Yaqi SHEN, Lianyungang (CN); Yue HU, Lianyungang (CN); Yanhua DING, Lianyungang (CN)

(21) Appl. No.: 18/832,422

(22) PCT Filed: Jan. 19, 2023
(86) PCT No.: PCT/CN2023/073126

§ 371 Date: Jul. 23, 2024

(30) Foreign Application Priority Data
Jan. 25, 2022 (CN) 202210087876.9

(51) Int. Cl. A61K 31/4155

(52) U.S. Cl. CPC A61K 31/4155

(57) ABSTRACT

The present disclosure relates to a dosage regimen for a thrombopoietin receptor agonist. In particular, the present disclosure relates to a method of administering hetrombopag or a pharmaceutically acceptable salt thereof to a patient in need, the patient suffering from mild liver injury or moderate liver injury.

### TECHNICAL FIELD

The present disclosure pertains to the technical field of pharmaceutics and relates to an administration regimen for a thrombopoietin receptor agonist.

### BACKGROUND

Platelets are cells that play an important role in hemostasis and the repair of damaged blood vessels. Clinically, thrombocytopenia in the patient can be caused by immune thrombocytopenia (ITP), bone marrow failure diseases such as aplastic anemia (AA), hematologic malignancies such as myelodysplastic syndromes (MDS), or chronic liver diseases, and by chemotherapy, radiation therapy, or bacterial or viral infections (HIV, HCV, etc.). The most significant dangers of thrombocytopenia are bleeding or increased risk of bleeding, which in severe cases can lead to visceral or even intracranial hemorrhage that endangers the patient's life, or limit clinical surgical treatment. Therefore, it is one of the hot spots of clinical research to rapidly, effectively, and safely increase the platelet count in patients with thrombocytopenic diseases. Studies find that a thrombopoietin receptor agonist (TPORA) has great significance in treating thrombocytopenia caused by various reasons.

The liver is a critical site for the biotransformation and elimination of drugs. Many reports in biomedical literature have confirmed that liver diseases can alter drug absorption and disposition (PK), as well as their effectiveness and safety (PD). Alterations in drug metabolism and excretion activities caused by hepatic functional impairments may lead to drug accumulation, or in rare cases, failure to form active metabolites, thereby affecting the drug's effectiveness and safety. A hepatic impairment is a condition in which the liver's normal function is diminished. A hepatic impairment can be acute, occurring rapidly, or chronic. Chronic hepatic impairments or cirrhosis can be caused by various factors, such as excessive alcohol consumption, hepatitis, autoimmune diseases, genetic or metabolic factors, or can be sudden. Hepatic impairments are usually irreversible. Treatment includes prevention of disease progression and treatment of symptoms. In severe cases, liver transplantation is the only option. Hepatic impairments do not show obvious symptoms, or they may be characterized by such symptoms as reduced blood clotting capacity (coagulopathy), brain dysfunction (encephalopathy), ascites, increased risk of infections, hypogonadism, changes in liver size, jaundice, and increased sensitivity to drugs.

Changes in the pharmacokinetic parameters (such as AUC, Cₘₐₓ, and t1/2) of drugs and/or their metabolites in patients with hepatic impairments can lead to numerous issues, including the need for dose adjustment, complicated physician prescribing, the need for liver function tests, the lack of availability of the correct dosage, the lack of availability of certain drugs for patients with hepatic impairments, and overdosing.

Overall, there are no clinically effective liver function indicators yet for predicting drug PK and PD. However, clinical studies conducted in patients with impaired liver function during drug development can provide some information that helps in determining the initial dose for these patients.

The present disclosure provides the compound represented by formula I ((Z)-5-(2-hydroxy-3-(2-(3-methyl-5-oxo-1-(5,6,7,8-tetrahydronaphthalen-2-yl)-1H-pyrazol-4(5H)-ylidene)hydrazino)phenyl)furan-2-carboxylic acid) or a pharmaceutically acceptable salt thereof, i.e., herombopag or a pharmaceutically acceptable salt thereof, which can be used as a thrombopoietin (TPO) receptor agonist for increasing the level of platelets in the blood to treat a variety of blood diseases, such as diseases caused by platelet defects. It can also be used for treating thrombocytopenia in patients caused by immune thrombocytopenia (ITP), bone marrow failure diseases such as aplastic anemia (AA), hematologic malignancies such as myelodysplastic syndromes (MDS), or chronic liver diseases, and by chemotherapy, radiation therapy, or bacterial or viral infections (HIV, HCV, etc.).

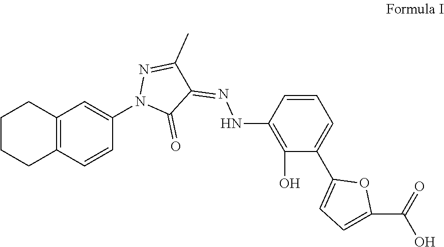 [SMILES: CC1=NN(c2ccc3c(c2)CCCC3)C(=O)/C1=N\Nc1cccc(-c2ccc(C(=O)O)o2)c1O]

### SUMMARY

The present disclosure provides a method for administering herombopag or a pharmaceutically acceptable salt thereof to a patient in need thereof, wherein the patient has a mild hepatic impairment; the method comprises:
administering to the patient having a mild hepatic impairment a therapeutically effective amount of herombopag or the pharmaceutically acceptable salt thereof, wherein an initial dose of the herombopag or the pharmaceutically acceptable salt thereof is unadjusted as compared to an initial dose administered to a patient having normal liver function.

The present disclosure also provides herombopag or a pharmaceutically acceptable salt thereof for use in a method for treating thrombocytopenia in a patient in need thereof, wherein the patient has a mild hepatic impairment; the method comprises: administering to the patient having a mild hepatic impairment a therapeutically effective amount of herombopag or the pharmaceutically acceptable salt thereof, wherein an initial dose of the herombopag or the pharmaceutically acceptable salt thereof is unadjusted as compared to an initial dose administered to a patient having normal liver function.

The present disclosure also provides use of herombopag or a pharmaceutically acceptable salt thereof for treating thrombocytopenia, wherein the patient has a mild hepatic impairment; a therapeutically effective amount of herombopag or the pharmaceutically acceptable salt thereof is administered to the patient having a mild hepatic impairment, wherein an initial dose of the herombopag or the pharmaceutically acceptable salt thereof is unadjusted as compared to an initial dose administered to a patient having normal liver function.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 2.5-15 mg of herombopag.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 2.5 mg of herombopag.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 5 mg of herombopag.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 7.5 mg of herombopag.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 10 mg of herombopag.

A method for administering herombopag or a pharmaceutically acceptable salt thereof to a patient in need thereof is provided, wherein the patient has a mild hepatic impairment; the method comprises: administering to the patient having a mild hepatic impairment the herombopag or the pharmaceutically acceptable salt thereof in an amount that is equivalent to 15 mg of herombopag.

The present disclosure provides a method for administering herombopag or a pharmaceutically acceptable salt thereof to a patient in need thereof, wherein the patient has a moderate hepatic impairment; the method comprises:
administering to the patient having a moderate hepatic impairment a therapeutically effective amount of herombopag or the pharmaceutically acceptable salt thereof, wherein an initial dose of the herombopag or the pharmaceutically acceptable salt thereof is less than an initial dose administered to a patient having normal liver function or a mild hepatic functional impairment.

The present disclosure also provides herombopag or a pharmaceutically acceptable salt thereof for use in a method for treating thrombocytopenia in a patient in need thereof, wherein the patient has a moderate hepatic impairment; the method comprises:
administering to the patient having a moderate hepatic impairment a therapeutically effective amount of herombopag or the pharmaceutically acceptable salt thereof, wherein an initial dose of the herombopag or the pharmaceutically acceptable salt thereof is less than an initial dose administered to a patient having normal liver function or a mild hepatic functional impairment.

The present disclosure also provides use of herombopag or a pharmaceutically acceptable salt thereof for treating thrombocytopenia, wherein the patient has a moderate hepatic impairment; a therapeutically effective amount of herombopag or the pharmaceutically acceptable salt thereof is administered to the patient having a moderate hepatic impairment, wherein an initial dose of the herombopag or the pharmaceutically acceptable salt thereof is less than an initial dose administered to a patient having normal liver function or a mild hepatic functional impairment.

In some embodiments, the method comprises the initial dose of the herombopag or the pharmaceutically acceptable salt thereof administered to the patient having a moderate hepatic impairment being 10%-90% less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 20%-80% less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 30%-70% less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 40%-60% less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 50% less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 1 mg-5 mg less than the initial dose administered to the patient having normal liver function or a mild hepatic functional impairment.

In some other embodiments, the method comprises the initial dose of the herombopag or the pharmaceutically acceptable salt thereof administered to the patient having a moderate hepatic impairment being 2.5 mg less than the initial dose administered to the patient having normal liver function or a mild hepatic functional impairment.

A method for administering herombopag or a pharmaceutically acceptable salt thereof to a patient in need thereof is provided, wherein the patient has a moderate hepatic impairment; the method comprises: administering to the patient having a moderate hepatic impairment the herombopag or the pharmaceutically acceptable salt thereof in an amount that is equivalent to 2.5-15 mg of herombopag.

A method for administering herombopag or a pharmaceutically acceptable salt thereof to a patient in need thereof is provided, wherein the patient has a moderate hepatic impairment; the method comprises: administering to the patient having a moderate hepatic impairment the herombopag or the pharmaceutically acceptable salt thereof in an amount that is equivalent to 2.5 mg of herombopag.

A method for administering herombopag or a pharmaceutically acceptable salt thereof to a patient in need thereof is provided, wherein the patient has a moderate hepatic impairment; the method comprises: administering to the patient having a moderate hepatic impairment the herombopag or the pharmaceutically acceptable salt thereof in an amount that is equivalent to 5 mg of herombopag.

A method for administering herombopag or a pharmaceutically acceptable salt thereof to a patient in need thereof is provided, wherein the patient has a moderate hepatic impairment; the method comprises: administering to the patient having a moderate hepatic impairment the herombopag or the pharmaceutically acceptable salt thereof in an amount that is equivalent to 7.5 mg of herombopag.

A method for administering herombopag or a pharmaceutically acceptable salt thereof to a patient in need thereof is provided, wherein the patient has a moderate hepatic impairment; the method comprises: administering to the patient having a moderate hepatic impairment the herombopag or the pharmaceutically acceptable salt thereof in an amount that is equivalent to 10 mg of herombopag.

The present disclosure also provides a method for administering herombopag or a pharmaceutically acceptable salt thereof to a patient in need thereof; administering a dose of the herombopag or the pharmaceutically acceptable salt thereof to a patient having a mild, moderate, or severe hepatic impairment results in higher exposure than administering the dose to a patient having normal liver function.

In certain embodiments, the exposure is measured by Cₘₐₓ or AUC_{0-∞}.

A method for administering herombopag or a pharmaceutically acceptable salt thereof to a patient in need thereof is provided, wherein the patient has a mild hepatic impairment; the method comprises: administering to the patient having a mild hepatic impairment a therapeutically effective amount of the herombopag or the pharmaceutically acceptable salt thereof, wherein administering herombopag to the patient having a mild hepatic impairment results in an AUC_{0-∞} that is 10-50% greater than that resulting from administering herombopag to a patient having normal liver function; preferably, the herombopag AUC_{0-∞} is 15%-30% greater; more preferably, the herombopag AUC_{0-∞} is 20-25% greater.

A method for administering herombopag or a pharmaceutically acceptable salt thereof to a patient in need thereof is provided, wherein the patient has a mild hepatic impairment; the method comprises: administering to the patient having a mild hepatic impairment a therapeutically effective amount of the herombopag or the pharmaceutically acceptable salt thereof, wherein administering herombopag to the patient having a mild hepatic impairment results in an AUC_{0-∞} that is within the range of 100%-125% as compared to a patient having normal liver function.

A method for administering herombopag or a pharmaceutically acceptable salt thereof to a patient in need thereof is provided, wherein the patient has a mild hepatic impairment; the method comprises: administering to the patient having a mild hepatic impairment a therapeutically effective amount of the herombopag or the pharmaceutically acceptable salt thereof, wherein administering herombopag to the patient having a mild hepatic impairment results in a Cₘₐₓ that is 10%-100% greater than that resulting from administering herombopag to a patient having normal liver function; preferably, the herombopag Cₘₐₓ is 30%-100% greater; more preferably, the herombopag Cₘₐₓ is 50%-100% greater; most preferably, the herombopag Cₘₐₓ is 60%-80% greater.

A method for administering herombopag or a pharmaceutically acceptable salt thereof to a patient in need thereof is provided, wherein the patient has a moderate hepatic impairment; the method comprises: administering to the patient having a moderate hepatic impairment a therapeutically effective amount of the herombopag or the pharmaceutically acceptable salt thereof, wherein administering herombopag to the patient having a moderate hepatic impairment results in an AUC_{0-∞} that is 30%-130% greater than that resulting from administering herombopag to a patient having normal liver function; preferably, the herombopag AUC_{0-∞} is 30%-100% greater; more preferably, the herombopag AUC_{0-∞} is 50%-100% greater; most preferably, the herombopag AUC_{0-∞} is 60%-80% greater.

A method for administering herombopag or a pharmaceutically acceptable salt thereof to a patient in need thereof is provided, wherein the patient has a moderate hepatic impairment; the method comprises: administering to the patient having a moderate hepatic impairment a therapeutically effective amount of the herombopag or the pharmaceutically acceptable salt thereof, wherein administering herombopag to the patient having a moderate hepatic impairment results in a Cₘₐₓ that is 50%-150% greater than that resulting from administering herombopag to a patient having normal liver function; preferably, the herombopag Cₘₐₓ is 70%-150% greater; more preferably, the herombopag Cₘₐₓ is 70%-130% greater; most preferably, the herombopag Cₘₐₓ is 90%-110% greater.

In certain embodiments, the method also comprises determining whether the patient has a mild, moderate, or severe hepatic impairment.

In certain embodiments, the herombopag or the pharmaceutically acceptable salt thereof is herombopag diethanolamine salt.

In certain embodiments, the herombopag or the pharmaceutically acceptable salt thereof is orally administered.

In certain embodiments, the herombopag or the pharmaceutically acceptable salt thereof is administered at a dose of 1-30 mg. The dose administered is the weight of herombopag in a single dose of herombopag or the pharmaceutically acceptable salt thereof. The dose administered is preferably 1.0 mg, 2.0 mg, 2.5 mg, 5 mg, 7.5 mg, 10 mg, 12.5 mg, 15 mg, 20 mg, 25 mg, or 30 mg.

In certain embodiments, herombopag or the pharmaceutically acceptable salt thereof is administered once daily, twice daily, or three times daily; preferably once daily.

The patients described in the present disclosure are human patients, including adult patients and pediatric patients.

The patients described in the present disclosure are in need of administration of the herombopag or the pharmaceutically acceptable salt thereof to achieve and maintain a platelet count of ≥50×10⁹/L.

Specifically, the method for administering herombopag or a pharmaceutically acceptable salt thereof to a patient in need thereof described in the present disclosure is a method for administering herombopag or a pharmaceutically acceptable salt thereof to treat thrombocytopenia in the patient in need thereof; the thrombocytopenia is selected from the group consisting of thrombocytopenia in the patient caused by immune thrombocytopenia, aplastic anemia, severe aplastic anemia, chronic liver diseases, or chemotherapy.

In certain embodiments, the herombopag or the pharmaceutically acceptable salt thereof described in the present disclosure is administered in the form of a pharmaceutical composition.

In certain embodiments, the composition described in the present disclosure comprises a therapeutically effective amount of herombopag or the pharmaceutically acceptable salt thereof and a pharmaceutically acceptable carrier.

In certain embodiments, the weight of herombopag in the pharmaceutical composition comprising herombopag or the pharmaceutically acceptable salt thereof described in the present disclosure is selected from the group consisting of 1.0 mg, 2.0 mg, 2.5 mg, 5 mg, 7.5 mg, 10 mg, 12.5 mg, 15 mg, 20 mg, 25 mg, and 30 mg.

Herombopag or the pharmaceutically acceptable salt thereof, the composition thereof, or the like may be prepared according to the patent applications WO2009092276, WO2010142137, WO2017124983, and WO2020151728, the disclosures of which are incorporated herein by reference in their entirety.

“Thrombocytopenia” described in the present disclosure includes, but is not limited to, thrombocytopenia caused by immune thrombocytopenia (ITP), bone marrow failure diseases such as aplastic anemia (AA), hematologic malignancies such as myelodysplastic syndromes (MDS), or chronic liver diseases, and by chemotherapy, radiation therapy, or bacterial or viral infections (HIV, HCV, etc.).

The herombopag described in the present disclosure has tautomers. Although the structural formula of herombopag is drawn as certain isomeric forms for the sake of simplicity, the present disclosure can include all isomers such as tautomers, rotamers, geometric isomers, diastereomers, racemates, and enantiomers. The tautomers of herombopag also fall within the protection scope of the present disclosure, for example:

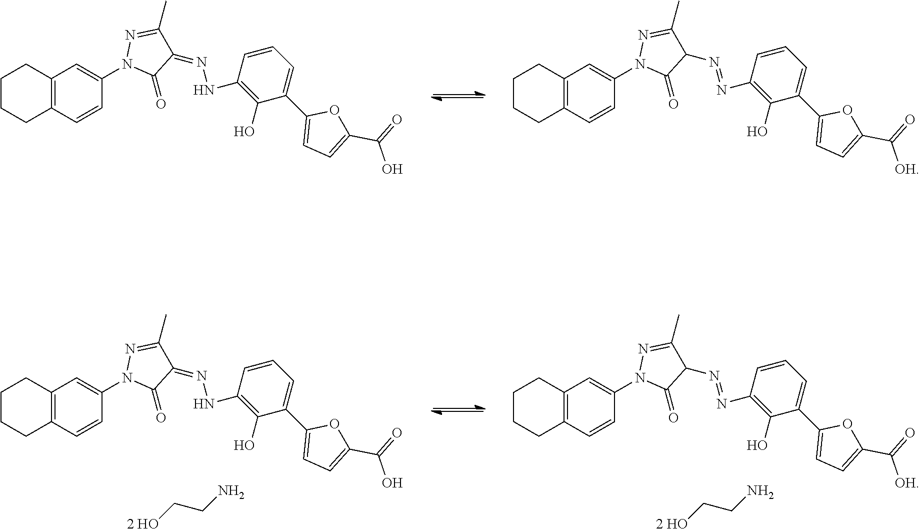

As described in the present disclosure, “adjusting administration”, “altering administration”, “adjusting dosing”, and “altering dosing” are all equivalent and mean tapering off, reducing, or increasing the dose of the substance, ceasing to administer the substance to the patient, or substituting a different active agent for the substance.

Reference throughout the specification to “one embodiment” or “an embodiment” or “some embodiments” or “a certain embodiment” means that a particular feature, structure, or characteristic described in connection with the embodiment is included in at least one embodiment. Thus, the appearances of the phrases “in one embodiment” or “in an embodiment” or “in some embodiments” or “in a certain embodiment” in various places throughout the specification do not necessarily all refer to the same embodiment. Furthermore, the particular features, structures, or characteristics may be combined in any suitable manner in one or more embodiments.

As described in the present disclosure, “hepatic impairment” means hepatocellular (liver) dysfunction.

As described in the present disclosure, “Child-Pugh score” is a score based on five clinical measures of hepatic impairment, including levels of total bilirubin, serum albumin, PT INR, ascites, and hepatic encephalopathy. Each measure has a grade of 1, 2, or 3, and the sum of the five grades is the Child-Pugh score. By placing subjects in a Child-Pugh group, the Child-Pugh score can be used to classify hepatic impairments.

As described in the present disclosure, “mild hepatic impairment” refers to a grade of the level of hepatic impairment based on a Child-Pugh score of 5-6.

As described in the present disclosure, “moderate hepatic impairment” refers to a grade of the level of hepatic impairment based on a Child-Pugh score of 7-9.

As described in the present disclosure, “severe hepatic impairment” refers to a grade of the level of hepatic impairment based on a Child-Pugh score of 10-15.

As described in the present disclosure, “hepatic impairment” and “hepatic insufficiency” have the same definition and are both defined based on the Child-Pugh scoring criteria below.

| | | Score of degree of abnormality | | |
|---|---|---|---|---|
| Clinical or biochemical measure | | 1 | 2 | 3 |
| Hepatic encephalopathy (grade)* | | 0 | 1 or 2 | 3 or 4 |
| Ascites | | None | Mild | Moderate |
| | | | | to severe |
| Serum total | mg/dL | <2 | 2~3 | >3 |
| bilirubin | μmol/L# | <34.2 | 34.2~51.3 | >51.3 |
| Serum albumin | g/dL | >3.5 | 2.8~3.5 | <2.8 |
| | g/L# | >35 | 28~35 | <28 |

| | | | |
|---|---|---|---|
| Prolonged prothrombin | <4 | 4-6 | >6 |
| time (seconds) | | | |

Child Pugh grades: mild: grade A, a score of 5-6; moderate: grade B, a score of 7-9; severe: grade C, a score of 10-15.
*hepatic encephalopathy grades:
grade 0: consciousness, personality, nervous system examination results, and electroencephalogram results are all normal;
grade 1: restlessness, sleep disorder, irritability/agitation, tremor, poor handwriting, electroencephalogram 5 cps waveform;
grade 2: lethargy, temporal perception disorder, abnormal behavior, asterixis, ataxia, electroencephalogram slow triphasic waves;
grade 3: drowsiness, sleepiness, disorientation, hyperreflexia, rigidity, electroencephalogram slow waves;
grade 4: coma from which the patient cannot be awakened, absence of personality/actions, absence of cerebral reflexes, electroencephalogram slow 2-3 cps δ wave activity.
#: The units of serum total bilirubin (mg/dL) and serum albumin (g/dL) are converted to μmol/L and g/L, respectively, with the conversion factors being 17.1 and 10, respectively.

As described in the present disclosure, “AUC” refers to the area under the curve or the integral of the plasma concentration of an active pharmaceutical ingredient or metabolite over time following an administration event.

As described in the present disclosure, “AUC₀₋ₜ” is the integral under the plasma concentration curve from time 0 (administration) to time “t”.

As described in the present disclosure, “AUC_{0-∞}” is the AUC from time 0 (administration) to time infinity. Unless otherwise stated, AUC refers to AUC0-∞. For example, a drug is packaged in a salt form, for example, herombopag diethanolamine salt, and the dosage form or dose refers to the equivalent mass of the corresponding free base (herombopag).

As described in the present disclosure, Cmax is a pharmacokinetic parameter and refers to the maximum plasma concentration observed after the delivery of an active pharmaceutical ingredient. Cmax occurs at the time of the maximum plasma concentration, tmax.

The AUC or Cₘₐₓ described in the present disclosure may be determined by routine experimentation in the art, or reference may be made to the descriptions in the examples.

The numerical values described in the present disclosure are calculated or inferred values with some errors, which means ±20% of the values and more specifically include ±10%, ±5%, ±2%, and ±1% of the values.

As described in the present disclosure, “administering to a patient” refers to the process of introducing a compound or a pharmaceutically acceptable salt, composition, or dosage form thereof into the patient by an art-recognized means of introduction.

As described in the present disclosure, “effective amount” and “therapeutically effective amount” of an agent, compound, drug, composition, or combination is an amount that is non-toxic and effective for producing some desired therapeutic effect upon administration to a subject or patient (e.g., a human subject or patient). The precise therapeutically effective amount for a subject may depend on, e.g., the subject's constitution and health, the nature and extent of the condition, the therapeutic agents or combination of therapeutic agents selected for administration, and other variables known to those skilled in the art. The effective amount for a given situation is determined by routine experimentation and is within the judgment of the clinician.

In the present disclosure, the initial dose is not exactly the same as a dose between therapeutically effective amounts, and the dose described by a therapeutically effective amount may be the initial dose, or may be the dose after monitoring and dose adjustment, and may be specifically an adjusted dose after monitoring of the platelet count or metabolism.

### DETAILED DESCRIPTION

### Example 1: Study of Pharmacokinetics and Safety of Trial Drug in Subjects with Mild Hepatic Impairments (Child-Pugh: Grade A), Moderate Hepatic Impairments (Child-Pugh: Grade B), and Normal Liver Function

### 1. Trial Drug

Trial drug: tablets of herombopag diethanolamine salt, produced and provided by Jiangsu Hengrui, strengths: 2.5 mg/tablet and 5 mg/tablet.

### 2. Inclusion Criteria

To be eligible for participation in this study, a subject must meet all of the following inclusion criteria:
1. Prior to the trial, the subject must sign a consent form, have a comprehensive understanding of the trial's content, process, and potential adverse reactions, and be capable of completing the study in accordance with the trial protocol;
2. The subject (and their partner) must be willing to take effective contraception measures from the time of screening to 6 months after the last dose of the study drug, as detailed in Appendix 1;
3. Age between 18 and 65 years old (inclusive), open to both sexes;
4. Body mass index (BMI=weight (kg)/height2 (m2)): 18-30 kg/m2 (inclusive);
5. Subjects with normal liver function must have normal clinical laboratory test results (complete blood count, blood biochemistry, urinalysis, and blood coagulation function) or the abnormalities in the results are of no clinical significance;
6. Subjects with normal liver function must have no history of serious primary diseases in major organs, including but not limited to gastrointestinal, respiratory, renal, hepatic, nervous, hematological, endocrine, oncological, immune, mental, or cardiovascular and cerebrovascular diseases.
   For Subjects with Hepatic Impairments, the Following Inclusion Criteria Must Also be Met:
7. No medication use within the 4 weeks prior to screening; or for those with hepatic functional impairments and/or other comorbid diseases that require long-term treatment, they must have been on stable medication for at least 4 weeks;
8. Patients with Child-Pugh grade A or B hepatic insufficiency caused by previous primary hepatic diseases (excluding drug-induced hepatic impairments).

### 3. Exclusion Criteria

If a subject meets any one of the following criteria, they will be excluded from this study:
1. Those who smoked, on average, more than 5 cigarettes per day within the 3 months prior to screening;
2. Those who have allergic diatheses or are allergic to any ingredients in herombopag diethanolamine tablets;
3. Those who consumed alcohol in excess of the following levels within the 3 months prior to screening: on average, more than 15 g per day for females (e.g., 145 mL of wine, 497 mL of beer, or 43 mL of low-alcohol liquor), and more than 25 g per day for males (e.g., 290 mL of wine, 994 mL of beer, or 86 mL of low-alcohol liquor);
4. Those who have a history of drug abuse within the 3 months prior to screening;
5. Those who donated or lost >400 mL of blood or received a blood transfusion within the 3 months prior to screening;
6. Those who underwent major surgery within the 6 months prior to screening or have surgical incisions that have not fully healed;
7. Those who have a history of deep vein thrombosis or other thromboembolic events or clinical symptoms suggesting thrombophilia;
8. Subjects who used TPO receptor agonists (such as eltrombopag and romiplostim) or Tpiao within the 1 month prior to screening;
9. Those who took herbal medicine or any drug that affects herombopag PK within the 14 days prior to taking the study drug (see Appendix 2 for drug interaction assessment);
10. Hypertension [systolic blood pressure (SBP) ≥160 mmHg and (or) diastolic blood pressure (DBP) ≥100 mmHg, confirmed once by retesting];
11. Female subjects who are breastfeeding or have positive serum pregnancy test results during screening or the trial;
12. Those with clinically significant abnormalities in 12-ECG (such as tachycardia/bradycardia that requires medication, second-third degree atrioventricular block, or QTcF interval prolongation [males: 470 ms; females: 480 ms](corrected using Fridericia's formula) or other abnormalities
deemed clinically significant by a clinician); 13. Those who have an estimated glomerular filtration rate (eGFR) of <60 mL/min/1.73 m2, as calculated using the modification of diet in renal disease (MDRD) formula;
14. Those who have malignant tumors or, within the 5 years prior to screening, a history of malignant tumors (excluding cutaneous non-melanoma that has been treated and has no signs of recurrence, and excised cervical intraepithelial neoplasia);
15. For subjects with normal liver function: those who participated in clinical trials of any drug or medical device within the 3 months prior to screening; for subjects with hepatic impairments: those who participated in clinical trials of any drug or medical device within the 1 month prior to screening;
16. For subjects with normal liver function: those who test positive for any one of the following indicators: hepatitis B surface antigen, hepatitis C antibodies or hepatitis C core antigen, HIV antibodies, or syphilis antibodies;
17. Those who are anticipated to tend to possibly require surgery or hospitalization during the trial;
18. Those who consumed any food or beverages containing alcohol (or had positive alcohol breath test results), grapefruit juice, or methylxanthines (such as coffee, tea, cola, chocolate, or functional beverages), or engaged in strenuous exercise and had other factors that affect the absorption, distribution, metabolism, or excretion of drugs, within the 1 day prior to administration;
19. Those who test positive for drugs (morphine, marijuana) in urine;
20. Those who are deemed by the researcher to have any factor that makes them unsuitable for participation in this trial.
   Additional Exclusion Criteria for Subjects with Hepatic Impairments (if a Subject Meets One of them, they Will be Excluded):
21. Those who have a history of liver transplantation;
22. Liver failure subjects, or cirrhosis subjects with complications such as hepatic encephalopathy, hepatocellular carcinoma, or esophagogastric variceal bleeding who are deemed unsuitable for participation in this study by the researcher;
23. Those who have, in addition to primary liver diseases, a history of any serious disease, or a history of diseases that are deemed by the researcher to possibly affect trial results or and/clinically significant abnormalities clinical laboratory examinations, or a history of diseases, including but not limited to diseases of the circulatory system, the endocrine system, the nervous system, the digestive system, and the urinary system, or hematological, immune, mental, and metabolic diseases.
24. Those who test positive for HIV antibodies; if a subject tests positive for syphilis antibodies, an additional rapid plasma reagin (RPR) test should be run, and if the RPR test result is also positive, the subject should be excluded.

### 4. Trial Process

This study is a single-dose, open-label trial designed to evaluate the pharmacokinetics of herombopag in subjects with normal liver function, mild hepatic impairments, and moderate hepatic impairments. A total of three groups were designed and are shown in Table 1 below.

TABLE 1
Groups of subjects

| | | Number |
|---|---|---|
| Group | Subject type | of subjects |
| A | Subjects with mild hepatic impairments | 8 |
| | (Child-Pugh: grade A) | |
| B | Subjects with moderate hepatic impairments | 8 |
| | (Child-Pugh: grade B) | |
| C | Subjects with normal liver function | 8 |

The severity of the hepatic functional impairments in the subjects was assessed using the Child-Pugh score (mild: grade A, a score of 5 or 6; moderate: grade B, a score of 7-9).

In this study, the subjects from group A and group B were included first. Once the inclusion of the two groups was complete, the subjects with normal liver function (group C) were then included to match all the subjects with hepatic impairments. The matching criteria for the study population are described as follows:
The average weight of group C falls within the range of the average weight of all the subjects with hepatic impairments ±10 kg (inclusive);
The average age of group C falls within the range of the average age of all the subjects with hepatic impairments ±10 years (inclusive);
The number of subjects of each sex in group C is similar to the number of all the subjects with hepatic impairments (±1 subject/sex);
There is no matching for age, sex, or weight between group A and group B.

All the subjects in each group underwent PK, PD, and safety index collection following the administration of the study drug on D1 according to the trial process chart, and comparative analysis was then performed between groups.

### 5. Blood Sampling Time Points

PK blood sampling time points: Venous blood samples of about 4 mL were collected from each subject within the 1 h prior to administration and at 0.5, 1, 2, 4, 6, 7, 8, 10, 12, 24, 48, 72, 96, and 120 h after administration. Blood sampling time windows: within the 60 min prior to administration; ±2 min for the 0.5 h-2 h after administration; ±5 min for the 4 h-12 h; ±15 min for the 24 h-120 h after administration.

### 6. Method of Administration to Subjects

Prior to the day-1 administration (D-1), the subjects were required to fast for 10 h or more, though water intake was not prohibited. The following morning (D1), the subjects orally took, on an empty stomach, a 7.5 mg dose of herombopag diethanolamine tablets (comprised of one 2.5 mg tablet and one 5 mg tablet) with about 240 mL of warm water. Water intake was prohibited 1 h before and after the administration. The subjects had lunch about 4 h after the administration and had dinner about 10 h after the administration. The subjects were advised to remain seated within the 2 h after the administration and to avoid strenuous exercise and prolonged bed rest.

### 7. Calculation of Pharmacokinetic Parameters

Pharmacokinetic parameters of herombopag, including Tₘₐₓ, Cₘₐₓ, AUC₀₋ₜ, AUC_{0-∞}, t_{1/2}, V/F, and CL/F, were calculated according to the actual sampling time points using a non-compartmental model (NCA) on Phoenix WinNonlin 8.1. The definitions and calculation of pharmacokinetic parameters are shown in Table 2 below.

TABLE 2
The definitions and calculation methods of pharmacokinetic parameters

| Parameter | Definition | Calculation method |
|---|---|---|
| AUC₀₋ₜ | Area under plasma | Trapezoidal method (linear up log |
| | concentration-time curve (0 to | down) |
| | sampling time t of the last | |
| | measurable concentration) | |
| AUC_{0-∞} | Area under plasma | AUC_{0-∞} = AUC₀₋ₜ + Cₜ/λ_{z} |
| | concentration-time curve (0 to | (t represents the sampling time of the |
| | infinity) | last measurable plasma concentration; |
| | | Cₜ represents the last measurable |
| | | sample drug concentration; λ_{z} |
| | | represents the terminal elimination |
| | | rate constant) |
| Cₘₐₓ | Peak concentration | Expressed using measured values |
| CL/F | Apparent clearance rate | CL/F = dose administered/AUC_{0-∞} |
| Tₘₐₓ | Time to peak | Expressed using measured values |
| t_{1/2} | Elimination half-life | t_{1/2} = Ln(2)/λ_{z} |
| V/F | Apparent volume of distribution | V/F = CL/F/λ_{z} |

### 8. Pharmacokinetic Parameter Analysis

The descriptive statistical summary of the main PK parameters of herombopag in the subjects with mild hepatic impairments (Child-Pugh: grade A), moderate hepatic impairments (Child-Pugh: grade B), and normal liver function is shown in Table 3. The comparative analysis of the PK parameters of herombopag between the subjects with hepatic impairments and the subjects with normal liver function is shown in Table 4.

TABLE 3
A summary of the PK parameters of herombopag (PKPS)

| | Normal liver | Mild hepatic | Moderate hepatic |
|---|---|---|---|
| | function group | impairment | impairment |
| | (n = 8) | (n = 8) | (n = 8) |
| Cₘₐₓ (ng/mL) | 36.8(70.4) | 62.6(42.2) | 82.8(54.0) |
| AUC₀₋ₜ (ng/mL) | 874(98.3) | 1060(40.6) | 1580(56.3) |
| AUC_{0-∞-}(ng/mL) | 929(101.6) | 1120(41.5) | 1660(58.8) |
| Tₘₐₓ (hr) | 8.00(1.00~8.00) | 2.00(1.00~8.00) | 2.00(2.00~4.00) |
| t_{1/2} (hr) | 34.6 ± 11.9 | 32.2 ± 7.28 | 36.3 ± 11.9 |
| CL/F (L/h) | 8.07(101.6) | 6.72(41.5) | 4.66(58.8) |
| V/F (L) | 372(60.3) | 304(45.6) | 233(69.7) |

Note:
Tₘₐₓ is expressed using Median (Min-Max), t_{1/2} is expressed using Mean ± SD, and the others are expressed using GeoMean (% CVb).

TABLE 4
A comparative analysis of the PK parameters of herombopag between the subjects
with hepatic impairments and the subjects with normal liver function (PKPS)

| | PK parameter | | | | Ratio | 90% CI of |
|---|---|---|---|---|---|---|
| Comparison | (unit) | Group | n | GLSM | (%) | Ratio (%) |
| Mild vs. | Cₘₐₓ | Mild hepatic | 8 | 62.6 | 170.39 | (106.63, 272.27) |
| Normal | (ng/mL) | impairment group | | | | |
| | | Normal liver function | 8 | 36.8 | | |
| | | group | | | | |
| | AUC₀₋ₜ | Mild hepatic | 8 | 1060 | 121.73 | (69.04, 214.63) |
| | (ng*h/mL) | impairment group | | | | |
| | | Normal liver function | 8 | 874 | | |
| | | group | | | | |
| | AUC_{0-∞} | Mild hepatic | 8 | 1120 | 120.16 | (67.25, 214.70) |
| | (ng*h/mL) | impairment group | | | | |
| | | Normal liver function | 8 | 929 | | |
| | | group | | | | |
| Moderate vs. | Cₘₐₓ | Moderate hepatic | 8 | 75.3 | 204.72 | (123.49, 339.38) |
| Normal | (ng/mL) | impairment group | | | | |
| | | Normal liver function | 8 | 36.8 | | |
| | | group | | | | |
| | AUC₀₋ₜ | Moderate hepatic | 8 | 1500 | 171.21 | (93.26, 314.30) |
| | (ng*h/mL) | impairment group | | | | |
| | | Normal liver function | 8 | 874 | | |
| | | group | | | | |
| | AUC_{0-∞} | Moderate hepatic | 8 | 1610 | 173.06 | (92.66, 323.22) |
| | (ng*h/mL) | impairment group | | | | |
| | | Normal liver function | 8 | 929 | | |
| | | group | | | | |

Note:
GLSM stands for geometric least squares mean.

After the subjects with normal liver function, mild hepatic impairments (Child-Pugh: grade A), and moderate hepatic impairments (Child-Pugh: grade B) each orally took a single 7.5 mg dose of herombopag diethanolamine tables on an empty stomach, the medians of Tₘₐₓ were 8.00 h, 2.00 h, and 2.00 h, respectively; the geometric means of Cₘₐₓ were 36.8 ng/mL, 62.4 ng/mL, and 82.8 ng/mL, respectively; and the geometric means of AUC₀₋ₜ were 874 h*ng/mL, 1060 h*ng/mL, and 1580 h*ng/mL, respectively; the geometric means of AUC_{0-∞} were 929 h*ng/mL, 1120 h*ng/mL, and 1660 h*ng/mL, respectively; and the arithmetic means of t_{1/2} were 34.6 h, 32.3 h, and 36.3 h, respectively. Analysis of variance (ANOVA) results show that according to a comparison between the subjects with mild hepatic impairments and the subjects with normal liver function, the geometric least squares mean ratios (mild/normal) for Cₘₐₓ, AUC₀₋ₜ, and AUC_{0-∞}, as well as their 90% confidence intervals, are 170.39% (106.63%, 272.27%), 121.73% (69.04%, 214.63%), and 120.16% (67.25%, 214.70%), respectively. According to a comparison between the subjects with moderate hepatic impairments and the subjects with normal liver function, the geometric least squares mean ratios (moderate/normal) for Cₘₐₓ, AUC₀₋ₜ, and AUC_{0-∞}, as well as their 90% confidence intervals, are 204.72% (123.49%, 339.38%), 171.21% (93.26%, 314.30%), and 173.06% (92.66%, 323.22%), respectively.

According to a comparison between the subjects with mild hepatic impairments and the subjects with normal liver function, Cₘₐₓ was increased by 70.39%, AUC₀₋ₜ was increased by 21.73%, and AUC_{0-∞} was increased by 20.16%. According to a comparison between the subjects with moderate hepatic impairments and the subjects with normal liver function, Cₘₐₓ was increased by 104.72%, AUC₀₋ₜ was increased by 71.21%, and AUC_{0-∞} was increased by 73.06%. The groups exhibited substantially identical half-lives. The subjects from the hepatic impairment groups exhibited a smaller median time to peak than those from the normal liver function group.

### 9. Conclusion

According to a comparison between the subjects with mild hepatic impairments and the subjects with normal liver function, Cₘₐₓ was increased by 70.39%, AUC₀₋ₜ was increased by 21.73%, and AUC_{0-∞} was increased by 20.16%. According to a comparison between the subjects with moderate hepatic impairments and the subjects with normal liver function, Cₘₐₓ was increased by 104.72%, AUC₀₋ₜ was increased by 71.21%, and AUC_{0-∞} was increased by 73.06%.

Hepatic impairments did not significantly affect the plasma protein binding rate of herombopag.

After the administration, all the groups showed a trend towards higher platelet counts compared to the baseline, and no significant differences were observed.

In the subjects with normal liver function and the subjects with mild and moderate hepatic impairments, a single oral dose of herombopag was safe and well tolerated. As the severity of liver impairment increased, the incidence of adverse reactions also rose.

In this study, a total of 24 subjects were included, and all of them were included in a safety analysis set. The results of the study show that a single 7.5 mg oral dose of herombopag diethanolamine tablets was safe and well tolerated in the subjects from the three groups (the normal liver function group, the mild hepatic impairment group, and the moderate hepatic impairment group): there were no unexpected adverse reactions, no reports of SAEs, and no subjects withdrawing early from the trial due to AEs.

In this trial, a total of 15 subjects experienced adverse events (29 events, with an incidence of 62.5%), and 13 of them experienced AEs related to the study drug (25 events, with an incidence of 54.2%): 2 from the normal liver function group (2 events, with an incidence of 25.0%), 4 from the mild hepatic impairment group (5 events, with an incidence of 50.0%), and 7 from the moderate hepatic impairment group (18 events, with an incidence of 87.5%). As the severity of hepatic functional impairment increased, the incidence of adverse reactions also rose.

In this study, all drug-related AEs were graded according to CTCAE5.0. Only 1 subject from the moderate hepatic impairment group (12.5%) experienced grade 3 events, which were decreased white blood cell count, decreased lymphocyte count, decreased platelet count, and decreased neutrophil count. Seven subjects (29.2%) experienced grade 2 events: 1 from the normal liver function group (12.5%) experienced hypertriglyceridemia, 1 from the mild hepatic impairment group (12.5%) experienced decreased neutrophil count, and 5 from the moderate hepatic impairment group (62.5%) experienced events including decreased white blood cell count, decreased platelet count, decreased neutrophil count, and anemia. Nine subjects (37.5%) experienced grade 1 events: 1 from the normal liver function group (25.0%), 3 from the mild hepatic impairment group (37.5%), and 5 from the moderate hepatic impairment group (62.5%). The adverse reactions mainly occurred in the hepatic impairment groups, and the incidence of adverse reactions in the moderate hepatic impairment group was relatively high, which may be attributed to the fact that these subjects had pre-existing liver diseases of varying degrees of severity and most of them already had abnormal baseline laboratory test results. These subjects recovered from all the adverse events without intervention.

Based on the pharmacokinetic results of herombopag in the subjects with hepatic impairments and the subjects with normal liver function, it is recommended that patients with mild hepatic impairments take the same initial dose as those with normal liver function, those with moderate hepatic impairments take half the initial dose, and the patients be closely monitored during treatment.

1. A method for administering herombopag or a pharmaceutically acceptable salt thereof to a patient in need thereof, wherein the patient has a mild hepatic impairment; the method comprises: administering to the patient having a mild hepatic impairment a therapeutically effective amount of herombopag or the pharmaceutically acceptable salt thereof, wherein an initial dose of the herombopag or the pharmaceutically acceptable salt thereof is unadjusted as compared to an initial dose administered to a patient having normal liver function.

2. A method for administering herombopag or a pharmaceutically acceptable salt thereof to a patient in need thereof, wherein the patient has a moderate hepatic impairment; the method comprises: administering to the patient having a moderate hepatic impairment a therapeutically effective amount of herombopag or the pharmaceutically acceptable salt thereof, wherein an initial dose of the herombopag or the pharmaceutically acceptable salt thereof is less than an initial dose administered to a patient having normal liver function or a mild hepatic functional impairment.

3. The method according to claim 2, wherein the initial dose of the herombopag or the pharmaceutically acceptable salt thereof administered to the patient having a moderate hepatic impairment is 10%-90% less than the initial dose administered to the patient having normal liver function or a mild hepatic functional impairment.

4. The method according to claim 2, wherein the initial dose of the herombopag or the pharmaceutically acceptable salt thereof administered to the patient having a moderate hepatic impairment is 50% less than the initial dose administered to the patient having normal liver function or a mild hepatic functional impairment.

5. A method for administering herombopag or a pharmaceutically acceptable salt thereof to a patient in need thereof, wherein administering a dose of the herombopag or the pharmaceutically acceptable salt thereof to a patient having a mild, moderate, or severe hepatic impairment results in higher exposure than administering the dose to a patient having normal liver function.

6. The method according to claim 5, wherein the exposure is measured by Cₘₐₓ or AUC_{0-∞}.

7. The method according to claim 6, wherein
a therapeutically effective amount of the herombopag or the pharmaceutically acceptable salt thereof is administered to the patient having a mild hepatic impairment, wherein administering herombopag to the patient having a mild hepatic impairment results in an AUC_{0-∞} that is 10-50% greater than that resulting from administering herombopag to the patient having normal liver function.

8. The method according to claim 6, wherein: a therapeutically effective amount of the herombopag or the pharmaceutically acceptable salt thereof is administered to the patient having a moderate hepatic impairment, wherein administering herombopag to the patient having a moderate hepatic impairment results in an AUC_{0-∞} that is 30%-130% greater than that resulting from administering herombopag to the patient having normal liver function.

9. The method according to claim 1, wherein the patient is in need of administration of the herombopag or the pharmaceutically acceptable salt thereof to achieve and maintain a platelet count of ≥50×10⁹/L.

10. The method according to claim 1, wherein the herombopag or the pharmaceutically acceptable salt thereof is administered to the patient to treat thrombocytopenia.

11. The method according to claim 1 wherein the herombopag or the pharmaceutically acceptable salt thereof is herombopag diethanolamine salt.

12. The method according to claim 1, wherein the herombopag or the pharmaceutically acceptable salt thereof is administered at a dose of 1-30 mg.

13. The method according to claim 1, wherein the herombopag or the pharmaceutically acceptable salt thereof is administered in the form of a pharmaceutical composition.

14. The method according to claim 13, wherein the composition comprises a therapeutically effective amount of herombopag or the pharmaceutically acceptable salt thereof and a pharmaceutically acceptable carrier.

15. The method according to claim 2, wherein the initial dose of the herombopag or the pharmaceutically acceptable salt thereof administered to the patient having a moderate hepatic impairment is 20%-80% less than the initial dose administered to the patient having normal liver function or a mild hepatic functional impairment.

16. The method according to claim 2, wherein the initial dose of the herombopag or the pharmaceutically acceptable salt thereof administered to the patient having a moderate hepatic impairment is 30%-70% less than the initial dose administered to the patient having normal liver function or a mild hepatic functional impairment.

17. The method according to claim 6, wherein a therapeutically effective amount of the herombopag or the pharmaceutically acceptable salt thereof is administered to the patient having a mild hepatic impairment, wherein administering herombopag to the patient having a mild hepatic impairment results in an AUC_{0-∞} that is 15%-30% greater than that resulting from administering herombopag to the patient having normal liver function.

18. The method according to claim 2, wherein the patient is in need of administration of the herombopag or the pharmaceutically acceptable salt thereof to achieve and maintain a platelet count of ≥50×10⁹/L.

19. The method according to claim 1, wherein the herombopag or the pharmaceutically acceptable salt thereof is administered to the patient to treat thrombocytopenia, wherein the thrombocytopenia is selected from the group consisting of thrombocytopenia in the patient caused by immune thrombocytopenia, aplastic anemia, severe aplastic anemia, chronic liver diseases, or chemotherapy.

20. The method according to claim 1, wherein the herombopag or the pharmaceutically acceptable salt thereof is administered at a dose of 2.5 mg, 3.75 mg, 5 mg, 7.5 mg, 10 mg, 12.5 mg, or 15 mg.
